(19) United States
(12) Patent Application Publication
(10) Pub. No.: US 2010/0305366 A1
(43) Pub. Date: Dec. 2, 2010

(54) Lutein Extraction Processes

(76) Inventors: Juan Liu, Nanjing (CN); Shufeng Fan, Nanjing (CN)

Correspondence Address: THOMAS, KAYDEN, HORSTEMEYER & RISLEY, LLP, 600 GALLERIA PARKWAY, S.E., STE 1500, ATLANTA, GA, 30339-5994, US

(21) Appl. No.: 12/609,135

(22) Filed: Oct. 30, 2009

(30) Foreign Application Priority Data
Oct. 31, 2008 (CN) 200810195251.4

(51) Int. Cl. C07C 35/21

(52) U.S. Cl. 568/816

(57) ABSTRACT

A lutein extraction process comprises pulverizing marigold, adding organic solvent in the former, then placing into an ultrasonic extraction vessel for ultrasonic extraction, filtering the extracted solution, and recovering to obtain a filtrate containing lutein ester; adding an alkaline solution that is 2 times of the filtrate volume in the filtrate recovered in the above step, and performing saponification on the lutein ester in the filtrate at 50-70° C.; washing the resultant after saponification with deionized water until lutein crystallization occurs, centrifuging the solution containing the lutein crystals to obtain crude crystals of lutein; subjecting the crude crystals of lutein to recrystallization; washing the lutein crystals obtained by recrystallization, and drying to obtain lutein crystal product. The ultrasonic extraction method in the present invention utilizes ultrasonic wave-induced intensive vibration, high acceleration, intensive cavitation effect, and stirring action to accelerate entrance of lutein into solvent, so as to increase extraction rate of effective components and shorten the extraction time.

### CROSS REFERENCE TO RELATED APPLICATIONS

This application claims priority to co-pending Chinese Patent Application No. CN 200810195251.4 entitled “Extraction Process of Lutein”, filed Oct. 31, 2008.

### TECHNICAL FIELD

The preset invention relates to an extraction method for natural colorant, and more particularly relates to a lutein extraction process.

### TECHNICAL BACKGROUND

Lutein, also called “plant lutein,” is a carotenoid compound and a main constituent of plant colorant in corn, vegetables, fruits, and flowers, etc., and widely exists in plants like spinach, cabbage, peach, mango, papaya, banana, potato, chrysanthemum, and pot marigold, of which marigold has the highest lutein content and is a main raw material resource for lutein extraction. Lutein has a molecular formula of C₄₀H₅₆O₂, and a molecular weight of 568.85. Free lutein is extremely unstable to heat, light, and oxygen.

Lutein exists in many kinds of plants, and exerts protection on plant photosynthesis. As early as the mid-1980s, Western medical researchers had found that natural lutein contained in plants was an excellent antioxidant. A large amount of epidemiological evidence suggests that lutein protects vision, has effects in preventing cataract and arteriosclerosis, enhances immunity, and particularly has significant functions for preventing cancer formation and delaying cancer development. Lutein is nontoxic and harmless, and adding it in food at certain amounts can prevent diseases induced by human organ aging.

Conventional extraction methods for lutein mainly comprise organic solvent extraction, microwave heating, and supercritical carbon dioxide extraction. The organic solvent extraction method has a low extraction efficiency and a long extraction time; and due to poor stability of free lutein, long extraction times may cause oxidation and degradation of lutein. Additionally, the supercritical carbon dioxide extraction method has the disadvantages of high equipment investment and high production cost.

Ultrasonic waves are elastic mechanical waves in a substance medium, and generate mechanical vibration of particles in the substance medium. The interaction in the medium caused by energy-containing ultrasonic vibration comprises heat action, mechanical action, and cavitation action. Ultrasonic extraction techniques utilize the aforementioned effects to generate transient high-temperature and high-pressure fields in fluids, and thus exert powerful shearing forces on the solid surfaces in the fluids, accelerating dissolution of natural active components and enhancing the mass transfer process.

So far, no report and patent regarding lutein extraction using ultrasound has been published.

### SUMMARY

### Technical Problem

Aiming at above technical blank, the present invention provides an ultrasonic lutein extraction process, which avoids the adverse influence of solvent extraction on lutein stability, improves the extraction speed of lutein, and increases the extraction rate of effective components.

### Technical Scheme

A lutein extraction process comprises the following extraction steps: a. pulverizing marigold, and adding organic solvent in the former, then placing into an ultrasonic extraction vessel for ultrasonic extraction at a marigold flower mass-to-organic solvent volume ratio of 1:20-60, a temperature of 20-40° C., an ultrasonic power of 100-500 watts (W), and an extraction time of 10-60 minutes (min.), filtering the extractive solution, and recovering to obtain a filtrate containing lutein ester; b. adding an alkaline solution with 2 times the volume of the filtrate into the filtrate recovered in the step a, and performing saponification of lutein ester in the filtrate at 50-70° C. for 30-50 min.; c. washing the reaction products of saponification with deionized water until lutein crystallization occurs, centrifuging the solution containing the lutein crystals to obtain crude lutein crystals; d. subjecting the crude lutein crystals to recrystallization with a tetrafuran/deionized water system; and e. washing and drying the lutein crystals obtained by recrystallization to obtain a lutein crystal product.

The organic extraction solvent is a mixed solvent of n-hexane, acetone, and methanol at a volume ratio of 8:1:1.

The alkaline solution in step b is an ethanol solution with a 20% mass concentration of KOH.

The volume ratio of tetrafuran and deionized water in the tetrafuran/deionized water system is 3:5.

The washing liquid in step e is deionzed water or ethanol.

### BENEFITS

The ultrasonic extraction method for lutein in the present invention utilizes intensive vibration, high acceleration, intensive cavitation effect, and stirring action induced by ultrasonic waves to accelerate the entrance of lutein into solvent, so as to increase the extraction rate of effective components and shorten the extraction time. Compared with the supercritical carbon dioxide extraction method, the disclosed method possesses low equipment investment and simple processing. The ultrasonic extraction technique is a low-temperature physical extraction process, which is more beneficial for extraction of lutein with poor thermal stability. The process avoids contact with light to improve stability of the reaction product. Compared with other methods, the method has the advantages of convenient operation, time saving, high extraction efficiency, and high safety. As lutein is prone to oxidation, the ultrasound frequency and ultrasound treatment time are key factors in determining the result of the ultrasonic extraction process. The method strictly controls the two process parameters on the basis of prior art to reach optimal ultrasound frequency and ultrasonic treatment time, so as to achieve maximal extraction efficiency.

### DESCRIPTION OF DRAWINGS

FIG. 1 describes influence of the ratio of liquid to material on extraction efficiency. It can be observed from the figure that when the ratio of liquid to material (volume/mass ratio) is larger than 40, the extraction efficiency is reduced along with the increase of solvent amount, which shows that when ratio of liquid to material is 40, lutein and extraction solvent substantially reach dissolution balance.

FIG. 2 describes the influence of extraction time on extraction efficiency. It can be observed from the figure that when the extraction time is short, the dissolution balance of lutein and the extraction solvent is not yet reached, therefore the extraction rate is low; but when the extraction time is long, as lutein is unstable to heat, heat generated by ultrasound has a certain damaging effect on lutein. Therefore the extraction rate is reduced if the extraction time is longer than a certain time, and the extraction effect is optimal when the extraction time is 30 min.

FIG. 3 describes the influence of ultrasound power on extraction efficiency. It can be observed from the figure that when the ultrasound power is higher than 300 W, the extraction efficiency is reduced. This shows that optimal extraction effect is achieved when the ultrasound power is 300 W.

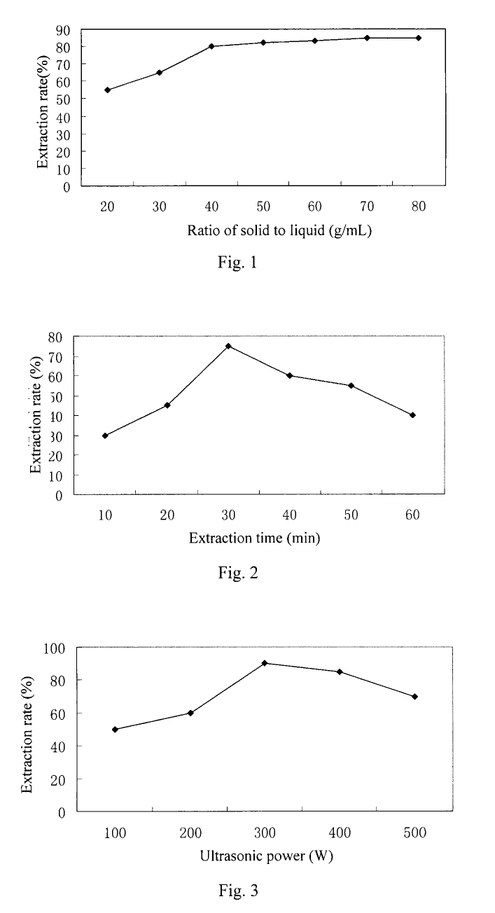

### DETAILED DESCRIPTION

### Embodiment 1

This embodiment comprises drying and pulverizing 1 kilogram (kg) of marigold, adding 20 liters (L) of a mixed solvent system of n-hexane/acetone/methanol (volume ratio of the three is 8:1:1), and ultrasonic extracting for 20 min. at 20° C. with an ultrasound power of 100 W; removing the above materials, placing them in centrifuge, and centrifuging at 5,000 revolutions per minute (rpm) to get a clear supernatant of lutein; adding an ethanol solution with a 20% mass concentration of KOH, which is 2 times of the supernatant volume, into the supernatant, and maintaining the temperature at 50-70° C. to carry out saponification for 30-50 min.; standing after saponification is completed, and cooling; washing the reaction product with deionized water until lutein crystallization and neutral, and centrifuging to get crude crystals of lutein; dissolving the crude crystals of lutein in 150 milliliter (mL) tetrafuran, adding 250 mL of deionized water, and recrystallizing; washing the lutein crystals obtained by recrystallization with deionized water and ethanol, and drying to obtain 3.58 grams (g) of lutein crystal product with an assay higher than 95% and a total lutein extraction rate of 93.99%.

### Embodiment 2

This embodiment comprises drying and pulverizing 1 kg marigold, adding 40 L of a mixed solvent system of n-hexane/acetone/methanol (volume ratio of the three is 8:1:1), and ultrasonic extracting at 30° C. for 30 min. with an ultrasound power of 300 W; removing the above materials, placing them in centrifuge, and centrifuging at 5,000 rpm to get a clear supernatant of lutein; adding an ethanol solution with a 20% mass concentration of KOH, which is 2 times of the supernatant volume, into the supernatant, and maintaining the temperature at 50-70° C. to carry out saponification for 30-50 min.; standing after saponification is completed, and cooling; washing the reaction product with deionized water until lutein crystallization and neutral, and centrifuging to get crude lutein crystals; dissolving the crude lutein crystals in 150 mL of tetrafuran, adding 250 mL of deionized water, and recrystallizing; washing the lutein crystals obtained by recrystallization with deionized water and ethanol, and drying to obtain 3.68 g of lutein crystal product with an assay higher than 95% and a total lutein extraction rate of 96.51%.

### Embodiment 3

This embodiment comprises drying and pulverizing 1 kg of marigold, adding 20 L of a mixed solvent system of n-hexane/acetone/methanol (volume ratio of the three is 8:1:1), and ultrasonic extracting at 40° C. for 30 min. with an ultrasound power of 300 W; removing the above materials, placing them in centrifuge, and centrifuging at 5,000 rpm to get a clear supernatant of lutein; adding an ethanol solution with a 20% mass concentration of KOH, which is 2 times of the supernatant volume, into the supernatant, and maintaining the temperature at 50-70° C. to carry out saponification for 30-50 min.; standing after saponification completed, and cooling; washing the reaction product with deionized water until lutein crystallization and neutral, and centrifuging to get crude crystals of lutein; dissolving the crude crystals of lutein in 150 mL of tetrafuran, adding 250 mL of deionized water, and recrystallizing; washing the lutein crystals obtained by recrystallization with deionized water and ethanol, and drying to obtain 3.66 g of crystal product of lutein with an assay higher than 95% and a total lutein extraction rate of 95.98%.

### Embodiment 4

This embodiment comprises drying and pulverizing 1 kg of marigold, adding 40 L of a mixed solvent system of n-hexane/acetone/methanol (volume ratio of the three is 8:1:1), and ultrasonic extracting at 40° C. for 40 min. with an ultrasound power of 500 W; removing the above materials, placing them in centrifuge, and centrifuging at 5,000 rpm to get a clear supernatant of lutein; adding an ethanol solution with a 20% mass concentration of KOH, which is 2 times of the supernatant volume, into the supernatant, and maintaining the temperature at 50-70° C. to carry out saponification for 30-50 min.; standing after saponification is completed, and cooling; washing the reaction product with deionized water until lutein crystallization and neutral, and centrifuging to get crude crystals of lutein; dissolving the crude crystals of lutein in 150 mL of tetrafuran, adding 250 mL of deionized water, and recrystallizing; washing the lutein crystals obtained by recrystallization with deionized water and ethanol, and drying to obtain 3.63 g of lutein crystal product with an assay higher than 95% and a total lutein extraction rate of 95.36%.

### Embodiment 5

This embodiment comprises drying and pulverizing 1 kg of marigold, adding 60 L mixed solvent system of n-hexane/acetone/methanol (volume ratio of the three is 8:1:1), and ultrasonic extracting at 20° C. for 40 min. with an ultrasound power of 500 W; removing the above materials, placing them in centrifuge, and centrifuging at 5,000 rpm to get a clear supernatant of lutein; adding an ethanol solution with a 20% mass concentration of KOH, which is 2 times of the supernatant volume, into the supernatant, and maintaining the temperature at 50-70° C. to carry out saponification for 30-50 min.; standing after saponification is completed, and cooling; washing the reaction product with deionized water until lutein crystallization and neutral, and centrifuging to get crude crystals of lutein; dissolving the crude crystals of lutein in 150 mL of tetrafuran, adding 250 mL of deionized water, and recrystallizing; washing the lutein crystals obtained by recrystallization with deionized water and ethanol, and drying to obtain 3.59 g of lutein crystal product with an assay higher than 95% and a total lutein extraction rate of 94.27%.

Comparison of Extraction Efficiencies

Extraction method

| | Solvent extraction | | | Ultrasonic extraction |
|---|---|---|---|---|
| | method | | | method |
| Times of | 1 | 2 | 3 | 1 |
| Extraction | | | | |
| Extraction | 45 | 63 | 70 | 95 |
| rate (%) | | | | |

The above table compares the extraction efficiencies of the ultrasonic extraction method and a standard organic solvent extraction method. It can be observed from the table that the ultrasonic extraction method can significantly improve the efficiency of extracting lutein from marigold. Maximal extraction efficiency for the solvent extraction method can only reach 70% after the extraction has been repeated three times, while the one-time extraction efficiency of the ultrasonic extraction method can reach 95%, which is significantly higher than the extraction efficiency of solvent extraction method.

1. A lutein extraction process, characterized in that the extraction procedure comprises:
a. pulverizing marigold and adding an organic solvent to form a marigold solution, placing the marigold solution into an ultrasonic extraction vessel, performing ultrasonic extraction on the marigold solution at a marigold mass-to-organic solvent volume ratio of 1:20-60, a temperature of 20-40° C., an ultrasonic power of 100-500 W, and an extraction time of 10-60 minutes to obtain an extracted solution, filtering the extracted solution, and recovering the extracted solution to obtain a filtrate containing lutein ester;
b. adding to the filtrate an alkaline solution that is twice the filtrate volume, and performing saponification on the lutein ester in the filtrate at 50-70° C. for 30-50 minutes to obtain a resultant product;
c. washing the resultant product with deionized water until lutein crystallization occurs and a crystal solution results, and centrifuging the crystal solution to obtain crude lutein crystals;
d. subjecting the crude lutein crystals to recrystallization with a mixed solvent system of tetrafuran/deionized water to obtain recrystallized lutein crystals;
e. washing the recrystallized lutein crystals, and drying the recrystallized lutein crystals to obtain a crystal product of lutein.

2. The lutein extraction process according to claim 1, characterized in that the organic solvent is a mixed solvent system of n-hexane, acetone, and methanol with a volume ratio of 8:1:1.

3. The lutein extraction process according to claim 1, characterized in that the alkaline solution is an ethanol solution with a 20% mass concentration of KOH.

4. The lutein extraction process according to claim 1, characterized in that the volume ratio of tetrafuran and deionized water in the mixed solvent system of the tetrafuran/deionized water is 3:5.

5. The lutein extraction process according to claim 1, characterized in that washing in step e comprises washing the recrystallized lutein crystals with deionized water or ethanol.
